(19) United States
(12) Patent Application Publication
(10) Pub. No.: US 2021/0299398 A1
(43) Pub. Date: Sep. 30, 2021

(54) BABY SLEEP AID USING VEHICLE MOTION STIMULATOR

(71) Applicants: SALUTARIS CONSORTIO, INC., Sunland, CA (US); AMERICAN INTERNATIONAL BUSINESS CONSULTANTS, LTD., Sunldand, CA (US)

(72) Inventor: Barry R. Chi, Tujunga, CA (US)

(21) Appl. No.: 17/136,027

(22) Filed: Dec. 29, 2020

Related U.S. Application Data
(60) Provisional application No. 63/002,294, filed on Mar. 30, 2020.

(51) Int. Cl. A61M 21/02; A47D 9/04; A47D 15/00

(52) U.S. Cl. CPC A61M 21/02; A61M 2021/0027; A47D 15/00; A47D 9/04

(57) ABSTRACT

A children sleep aid apparatus using a vehicle motion stimulator includes a sleep structure having a sleeping surface sized and configured for securely confining the children, a tactile transducer coupled to the sleeping structure, a vehicle simulation control unit in electrical communication with the tactile transducer, a loudspeaker and simple light projector. The vehicle simulation control unit comprises of a complex audio amplifier configured to generate sonic and tactile impulse patterns transmitted to the tactile transducer and loudspeaker. A light projector is mounted on the vehicle simulation unit. When activated, the unit produces electrical impulses to the tactile transducer, audible signals to the ambient speaker, and electrical power to the light projector to simulate tactile bumps and rumbles with the steady droning of moving car sounds and light patterns overhead simulating the experience of riding in a moving vehicle on a highway, with street lights rhythmically passing overhead.

This application claims the benefit of the filing date of provisional application No. 63/002,294, filed on Mar. 30, 2020.

### BACKGROUND

Motor vehicles are known to promote sleep in babies and infants when a motor vehicle is in steady motion for a period of time. This is principally due to the droning sound and vibration of the engine, various soft rumbling motions caused by the tires rolling over pavement surface irregularities and other surfaces, and in urban environments, the repeated exposure to external light sources as the vehicle passes them by. External light sources may include evenly spaced lamp lighting along freeways and highways, and/or external street lights, building lights and signage in an urban setting. It is the combined effect of light, sound, and motion that produces a calming effect in infants, babies, and small children, and promotes sleep rapidly and prolonged.

Since it is not practical or sometimes even feasible to arrange vehicle travel each time a parent or guardian desires to induce sleep for an infant, baby or small child, for napping or overnight purposes, there is a need for an apparatus and method that accomplishes this task while stationary, in an interior room, such as a bedroom, nursery, or other sleeping chamber.

It is therefore an object of the present disclosure to provide an apparatus and its method of use that reliably simulates the sensory environment of a moving vehicle without the need for such vehicle travel. Another object of the disclosure is to provide vehicle simulated stimulation in a non-moving setting, such as a bedroom or other stationary space for sleeping. It is another object of this present disclosure to provide an apparatus that produces motion, sound and light mimicking and characteristics of typical passenger vehicle travel. These and other objects of the disclosure are more fully discussed in the following specification, claims, and drawings.

### SUMMARY

The baby sleep aid apparatus uses simulated motion vehicle (such as a car or other motion or motor vehicle) stimulation to induce and promote the rapid onset of, and continued restful sleep in infants, babies, and small children (hereinafter “children”). The apparatus includes a sleeping structure having a sleeping surface sized and configured for securely confining children. The baby sleep aid apparatus includes a tactile transducer coupled to the sleeping structure such that it vibrates the sleeping surface, thereby mimicking the interior of a motorized vehicle, when running and in motion. In various implementations, the sleeping structure may be a structure chosen from the group consisting of a baby bed, a bassinet, a cot, a cradle, and/or a crib.

The baby sleep aid apparatus also includes a vehicle simulation control unit in electrical communication with the tactile transducer. In one implementation, the vehicle simulation control unit includes a complex audio amplifier, which is configured to generate tactile impulse patterns that are then transmitted to the tactile transducer, preferably using a wired communication, which may include powering the tactile transducer in some instances. The complex audio amplifier is preferably also in wired communication with a loud speaker, which may or may not be attached to or within the vehicle simulating control unit. When activated, the vehicle simulating control unit simultaneously causes the tactile transducer to produce motion in the form of vibrations at certain predetermined frequencies, and the loud speaker to produce gentle ambient sounds in the audible range.

The baby sleep aid apparatus also includes a simple light projector in electrical communication with the vehicle simulating control unit. In one implementation, the simple light projector is preferably mounted on the vehicle simulation control unit, and preferably on a flexible stalk configured for aiming the light in all directions. The simple light projector is configured to project a predetermined moving light pattern on nearby a ceiling or a wall when the vehicle simulator control unit is activated.

The baby sleep aid apparatus also includes a mounting apparatus configured for removably affixing the tactile transducer to a sleeping structure. In one implementation, the mounting apparatus includes a variable distance clamp bar, or other secure mounting feature, such that the produced tactile motions of the tactile transducer are transferred directly onto the sleeping structure, and thereby to the sleeping surface and infant sleeping thereon.

The tactile (i.e., vibration) transducer impulse patterns, and any desired ambient vehicle or other sounds, are generated by the complex audio amplifier that lies within the vehicle simulation control unit. The complex audio amplifier is configured to be able to modify the impulse and sound envelopes partners to the desired effect as dictated by the user. Moreover, the vehicle simulation control unit also provides an electrical signal to the simple light projector wherein the light pattern, such as simulating street lights rhythmically passed by a vehicle, may also be modified by the user.

For programmable control, the vehicle simulation control unit also preferably includes an SD card reader or similar memory, including removable memory, whereby a user may be able to upload additional/alternative vehicle motion simulation data into the vehicle simulation control unit so to produce motor vehicle environments with different characteristics. The control unit is preferably configured with an appropriate user interface for modifying relevant sound and motion parameters. In one implementation, the control unit includes the ability to accept an alternative vehicle simulation data stream which is pre-recorded by user (or created by a third party, including simulation data provided by the manufacturer) to provide a user with specific motor vehicle characteristics known to be effective.

In certain implementations, the vehicle simulation control unit may include a receiver that may be in wireless communication with user's mobile phone where an applicable mobile app downloaded to the user's mobile phone may remotely communicate and modify the signal output of the vehicle simulation control unit. In other implementations the vehicle simulation control unit may include a microphone, which may be an external microphone, configured to detect infant crying sounds with adjustable sensitivity, and automatic vibration, sound and light adjustment when infant crying sounds are detected. Additionally, the vehicle simulation control unit may be adjusted to automatically activate or turn on for a predetermined amount of time as set forth by the user to soothe infant/baby back to sleep, and the vehicle simulation control unit having an internal time clock where the duration of simulation effects may be adjusted by a user to shut off gradually over a certain time period.

### BRIEF DESCRIPTION OF THE FIGURES

FIG. 1 illustrates a baby sleep aid using simulated car stimulation apparatus.

FIG. 2 illustrates a tactile transducer affixed to a mounting apparatus for mounting the apparatus onto a sleep structure.

FIG. 3 illustrates attachment sites for the mounting apparatus.

FIG. 4 illustrates alternative attachment sites for the mounting apparatus.

FIG. 5 illustrates an additional alternative attachment site for the mounting apparatus.

FIG. 6 illustrates an additional alternative attachment site for the mounting apparatus.

FIG. 7 illustrates an additional alternative attachment site for the mounting apparatus.

FIG. 8 illustrates a schematic diagram of the second implementation baby sleep aid.

### REFERENCE NUMBERS

10. baby sleep aid apparatus
12. sleeping structure
14. sleeping surface
16. tactile transducer
18. vehicle simulation control unit
20. complex audio amplifier
22. speaker
24. mounting apparatus
26. first cord
27. second cord
28. first clamp assembly
30. second clamp assembly
32. bracket
34. wingnut
36. wingnut track
38. light source
40. flexible mount
46. power supply
48. SD card reader
50. microphone

### DESCRIPTION

Referring to FIG. 1, a baby sleep aid apparatus 10 using a motion vehicle stimulator to promote sleep in infants is shown. The baby sleep aid apparatus 10, includes a structure 12 having a sleeping surface 14 for an infant. In one implementation, the sleeping surface 14 is preferably sized and configured as a bed as illustrated. The sleeping structure 12 is sufficiently confining to maintain the infant in the bed sleeping structure 12. A tactile transducer 16 is coupled to the structure 12. The tactile transducer 16 is securely affixed to the sleeping structure 12, such that the vibrations, and tactile impulses produced by the tactile transducer 16 are transferred to the sleeping structure 12, thereby causing the sleeping surface 14 to gently vibrate and bump. The baby sleep aid apparatus 10 includes a light source 38, which may be a lamp or similar light producing apparatus. The light source 38 may be moving light pattern 42 that simulates street lamps as a moving vehicle travels under them. The baby sleep aid apparatus 10 may include a speaker wire 27 connecting the vehicle simulation control unit 18 to the speaker 22.

Still referring to FIG. 1, in the illustrated embodiment, the tactile transducer 16 is in wired communication with a vehicle simulation control unit 18. The vehicle simulation control unit 18 includes a complex audio amplifier 20, configured to generate tactile motions in low frequency impulses commonly known as rumbles and bumps. These tactile impulse patterns (not shown) are communicated to the tactile transducer 16, thereby causing the tactile transducer 16 to vibrate and bump according to predetermined but modifiable tactile patterns. The complex audio amplifier 20 has the capacity to modify the elements of the tactile wave envelope (i.e., attack, sustain, decay, release and frequency), including the intensity of the tactile wave itself.

The complex amplifier 20 within the vehicle simulation control unit 18 includes user interface control that allow users to shape the wave envelopes generated by the vehicle simulation control unit 18, which produces differing tactile motions and sound waves transmitted to the sleeping structure 12. Furthermore, the light patterns of the simple projector may also be modified by the vehicle simulation control unit 18. In one implementation, the vehicle simulation control unit 18 also includes wireless functionality and an associated processor to process and modify the sounds, the tactile motions, and the light patterns according to user preference. In other implementations, this may be accomplished remotely, by the use of user's mobile phone and appropriate mobile app.

The vehicle simulation control unit 18 is also preferably in wired communication with a speaker 22, using a second cord 27. Thus, when the vehicle simulation control unit 18 is activated, it simultaneously causes the tactile transducer 16 to produce tactile impulses in the predetermined modifiable frequencies, and also causes the speaker 22 to produce an accompanying sound. In one implementation, this may be an ambient internal motor vehicle sound in an audible range.

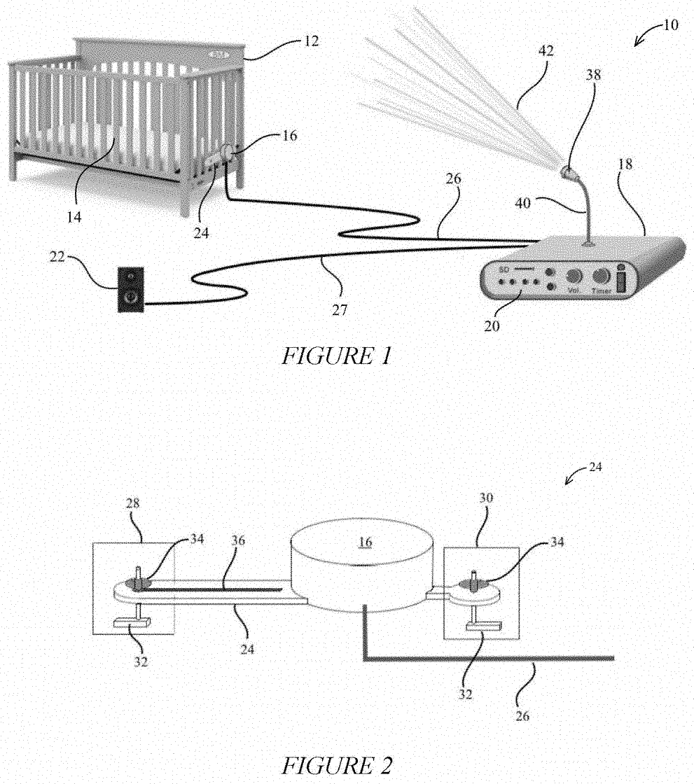

Referring to FIG. 2, the tactile transducer 16 is shown affixed to a mounting apparatus 24. The tactile transducer 16 is preferably enclosed in a magnetically shielded shell to avoid strong magnetic forces near the sleeping structure 12 and sleeping surface 14. Also shown in the illustrated view, a first cord 26 is shown connecting the tactile transducer 16 to the vehicle simulation control unit 18. The first cord 26 is preferably water proof and sufficiently resilient for repeated use.

The mounting apparatus 24, in the illustrated embodiment, comprises a substantially flat elongated member having a first clamp assembly 28 and a second clamp assembly 30 at either end. The mounting apparatus 24 with its elongated member is strong and inflexible in order to transfer all vibrations and tactile motions from the tactile transducer 16 to the sleeping structure 12 via the first clamp assembly 28 and the second clamp assembly 30. The first clamp assembly 28 and the second clamp assembly 30 each comprise a bracket 32 and a wingnut 34 for tightening the bracket against the sleeping structure 12 (not shown). A wingnut track 36 is provided for sliding the first clamp assembly 28 proximal or distal to the tactile transducer 16 in order to accommodate the mounting apparatus 24 onto differing sleep structure 12 dimensions.

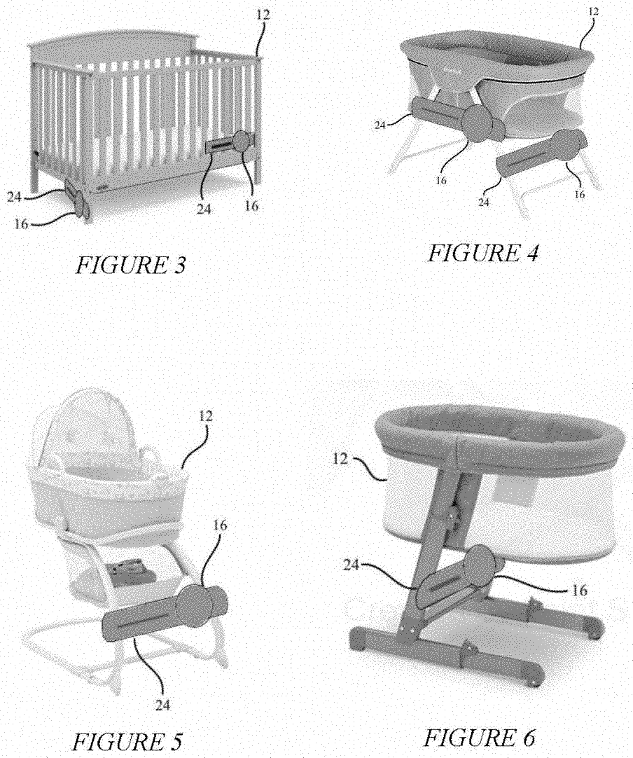

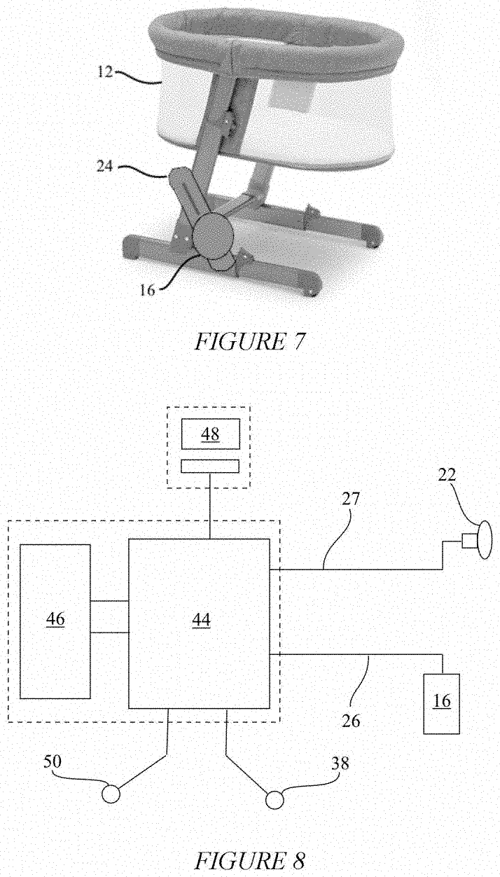

Referring to FIGS. 3-7 a variety of mounting positions for the mounting apparatus 24 on a variety of sleeping structures 12, such that the motion transducer 16 can impart vibrations to the sleeping structures 12 sufficiently to simulate the vibrations of a vehicle in motion.

Referring to FIG. 8, a diagrammatic representation of the complete baby sleep aid apparatus is shown. This includes the tactile transducer 16, the speaker 22, and the light source 38. Also shown is the vehicle simulation control unit 18, which includes an electronic control unit 44 and a power supply 46. Also shown in this view is an SD card reader 48 for receiving pre-recorded data stream of alternative vehicle riding characteristics (different tactile motions, sounds and lights). A microphone 50 (not shown) may also be included in the vehicle simulation control unit 18, and is configured to monitor and detect a child or infant's crying sounds. Once those sounds are detected, the vehicle simulation control unit 18 may be configured to automatically activate for a predetermined duration set by the user. In the illustrated implementation, the tactile transducer 16 and speaker 22 are physically independent structures from the vehicle simulation control unit 18, while the microphone (not shown) and the light source 38 share a common housing with the vehicle simulation control unit 18. In other implementations, these features may share a common housing with the vehicle simulation control unit 18. In virtually all implementations, the tactile transducer 16 is separate from the vehicle simulation control unit 18, to avoid damage from vibration, and to allow the vehicle simulation control unit 18 to be remote from the sleeping structure 12 (FIGS. 1, 3-7).

The foregoing description of the preferred embodiment of the Invention is sufficient in detail to enable one skilled in the art to make and use the invention. It is understood, however, that the detail of the preferred embodiment presented is not intended to limit the scope of the invention, in as much as equivalents thereof and other modifications which come within the scope of the invention as defined by the claims will become apparent to those skilled in the art upon reading this specification.

What is claimed is:

1. A children sleep aid apparatus using simulated motion vehicle stimulator, the apparatus comprising:
a structure having a sleeping surface sized and configured for securely confining the children; a tactile transducer coupled to the sleep structure;
a vehicle simulation control unit is in electrical communication with the tactile transducer;
the vehicle simulation control unit is comprised of a complex audio amplifier configured to generate low frequency impulse patterns and communicate the impulse patterns to the tactile transducer via a water resistant wired communication;
a loud speaker in electrical communication with the vehicle simulating control unit, wherein the vehicle simulation control unit, when activated, simultaneously causes the tactile transducer to produce tactile motions in predetermined, frequencies and the speaker to produce a gentle. internal moving motor vehicle sounds in audible range.

2. The baby sleep aid apparatus of claim 1 further comprising a simple light projector on the vehicle simulation control unit with a flexible stalk, the simple light projector is configured to project a predetermined light pattern.

3. The baby sleep aid apparatus of claim 2 wherein the vehicle simulation control unit causes the light source to project predetermined light patterns simultaneously with the tactile transducer producing vibration and tactile motion in certain low frequencies, and a loud speaker producing gentle ambient internal motor vehicles sounds in audible range.

4. The apparatus of claim 2 wherein the simple light projector is mounted on the vehicle simulation control unit.

5. The apparatus of claim 2, wherein the simple light projector comprises a flexible stalk configured for aiming the projector in multiple directions determined by the user.

6. The apparatus of claim 2, wherein the simple light projector is configured to project a moving light pattern.

7. The apparatus of claim 1, further comprising a mounting apparatus configured for removably affixing the tactile transducer to a sleeping structure.

8. The apparatus of claim 7, wherein the mounting apparatus comprises a variable distance clamp bar.

9. The apparatus of claim 1, wherein the impulse patterns generated by tactile transducer include periodic impulse (bump) patterns and random (steady state) vibrating patterns.

10. The apparatus of claim 1, wherein the vehicle simulation control unit comprised of a complex audio amplifier configured to have capabilities to generate and modify tactile and audio waves, and accept additional pre-recorded data streams of alternate motor vehicle environment via the SD card reader.

11. The apparatus of claim 1, wherein the vehicle simulation control unit includes an internal clock, the internal clock configured to automatically shut off the apparatus after a user-defined predetermined time.

12. The apparatus of claim 1, further comprising a wireless functionality where the vehicle simulation control unit is in wireless communication with the user's mobile phone via WiFi/Bluetooth connection where the user may remotely configure and govern the output signals of the vehicle control unit to modify the motion characteristics and activation duration of the baby sleep aid apparatus.

13. The baby sleep aid apparatus of claim 11, wherein the remote control is a device including wireless communication via WiFi/Bluetooth configured to communicate between the user's mobile phone with an appropriate network application, and the vehicle control unit.

14. The baby sleep aid apparatus of claim 1, wherein the sleep structure is a structure chosen from the group consisting of baby bed, bassinet, cot, cradle, and crib.

15. The vehicle simulation control unit of claim 1 further comprising a microphone configured to detect children crying sounds with adjustable sensitivity, and automatically activate the vehicle simulation control unit for a predetermined duration set by a user.

16. A method of producing a simulated vehicle tactile motions, sounds and lights simulating a riding environment is a baby sleep aid apparatus, the method comprising the steps of:
providing a structure having a sleeping surface and configuring the structure for securely confining the infant;
providing a vehicle simulation control unit having a complex audio amplifier with the capacity to generate and modify the output tactile impulses and audio signals to the tactile transducer and an ambient speaker;
providing a vehicle simulation control unit, and configuring the vehicle simulation control unit for transducer vibration adjustment control, transducer amplification, projector control, microphone functionality, WiFi functionality, SD card reading, and internal clock functionality;
providing a tactile transducer and coupling the tactile transducer to the sleeping structure;
further configuring the vehicle simulation control unit with complex audio amplifier functionality having the capacity to generate and modify the output tactile impulses and audio signals to the tactile transducer and an ambient speaker in tandem;
providing a tactile transducer and a speaker in electrical communication with the vehicle simulation control unit; and
activating the vehicle simulation control unit, whereby the tactile transducer produces a tactile motions and vibrations, and the speaker to producing ambient internal motor vehicle sounds in an audible range.

17. The method of claim 16, further comprising the step of providing a light projector in electrical communication with the vehicle simulation control unit, and configuring the light projector to project a predetermined light pattern.

18. The method of claim 16 further comprising the step of configuring the light projector to project predetermined light pattern simultaneously with the tactile vibrations and audible ambient sounds.

19. The method of claim 16 further comprising the step for providing a mounting apparatus wherein a tactile transducer is affixed and magnetically shielded, the mounting apparatus being sufficiently ridged to transfer all tactile motions from the tactile transducer to the sleeping structure, and having adjustable clamps to accommodate for the various widths of the sleep structure.

20. The method of claim 16 further comprising the step of configuring the tactile transducer to generate tactile impulse patterns and random vibrations.
